(19) United States
(12) Patent Application Publication
(10) Pub. No.: US 2021/0368861 A1
(43) Pub. Date: Dec. 2, 2021

(54) INFUSED SCENTED ESSENTIAL OIL BLOW THROUGH SHEETS

(71) Applicant: Chris Erin Laughlin, West Jordan, UT (US)

(72) Inventor: Chris Erin Laughlin, West Jordan, UT (US)

(21) Appl. No.: 15/929,930

(22) Filed: May 29, 2020

(51) Int. Cl. A24F 13/00; A24F 47/00; A61L 9/014; A61L 9/04

(52) U.S. Cl. CPC A24F 13/00; A24F 47/00; A61L 2209/14; A61L 9/042; A61L 9/014

(57) ABSTRACT

A material mass that is infused with essential oils, wherein the material mass is dimensioned and adapted for packing the material mass into a conduit for blowing smoke through, thereby covering up and/or removing the initial smell of the smoke and replacing the initial smell with an essential oil smell.

### CROSS-REFERENCE TO RELATED APPLICATION

This application claims the benefit of priority of U.S. provisional application No. 62/836,237, filed 19 Apr. 2019, the contents of which are herein incorporated by reference.

### BACKGROUND OF THE INVENTION

The present invention relates to smoking accessories and, more particularly, to an infused and/or scented essential oil blow through sheets. Specifically, the present invention relates to disposable blow through and filter agent sheet for marijuana cannabis smokers made of cloth and essential oil.

There are many medicinal and recreational smokers who are odor conscious and want to minimize the smell wherever they smoke for various reasons. Currently, such people use dryer sheets with a roll/tube (typically a toilet paper roll) to blow smoke through as a deodorizer, however dryer sheets have chemicals and toxins that may be harmful to accidently breath in during the process of minimizing the smell of smoke.

For instance, these makeshift solutions have a name, “sploofs”, which may be a plastic bottle with the end cut off, filled with dryer sheets, and covered with a thick sock, in some permutations. There are “commercial” sploofs on the market, but they are bulky and need to be replaced and do not have an essential oil component.

As can be seen, there is a need for a mass of infused scented essential oil blow material, possibly in the form of sheets, that can be packed into a conduit for blowing smoke through for the purpose of deodorizing. The present invention helps medicinal and recreational smokers who are odor conscious.

The present invention embodies essential oil sheets can be used in many different apparatuses. The sheets may be either infused with essential oil and/or scented with the essential oil in a such a way that smoke urged through such sheets have their initial odor rendered effectively odorless, as the scent/smell/odor of the essential oil overpowers the initial odor.

The essential oil sheets can be refills for pre-existing sploofs or other odor-minimizing solutions, and the essential oil sheets will be more affordable compared to currently solutions. Essential oil also has therapeutic benefits as well as having a pleasant smell. Disposable sheets can be replaced once the scent fades. Essential oil drops can be added to the disposable sheet for stronger odor. Infused sheets can also be reusable.

### SUMMARY OF THE INVENTION

In one aspect of the present invention, a refill element for a sploof for rendering an intrinsic smell of an exhale smoke odorless includes the following: a material mass infused with essential oil, wherein the material mass is cheese cloth, hemp, or bamboo, wherein the material mass is dimensioned to be a sheet, wherein the sheet is approximately eight by eight inches and has a thickness less than an eighth of an inch; and further include a plurality of sheets in a roll condition or disposed in a stacked condition, wherein each two adjacent sheet are interleaved.

In another aspect of the present invention, method of rending an intrinsic smell of an exhale smoke odorless includes the following: providing a material mass infused with essential oil; packing said material mass in a conduit; and exhaling through one end of the conduit, wherein the material mass is cheese cloth, hemp, or bamboo, wherein the material mass is dimensioned to be a sheet, wherein the sheet is approximately eight by eight inches and has a thickness less than an eighth of an inch; and further including either a plurality of sheets a roll condition; or disposed in a stacked condition, wherein each two adjacent sheet are interleaved.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention;

FIG. 2 is a perspective view of an exemplary embodiment of the present invention, shown in preparation of use;

FIG. 3 is a section view of an exemplary embodiment of the present invention, taken along line 3-3 in FIG. 2, and shown in use; and

FIG. 4 is a perspective view of an exemplary embodiment of the present invention, shown in use.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a material mass that is infused with essential oils, wherein the material mass is dimensioned and adapted for packing the material mass into a conduit for blowing smoke through, thereby covering up and/or removing the initial smell of the smoke and replacing the initial smell with an essential oil smell.

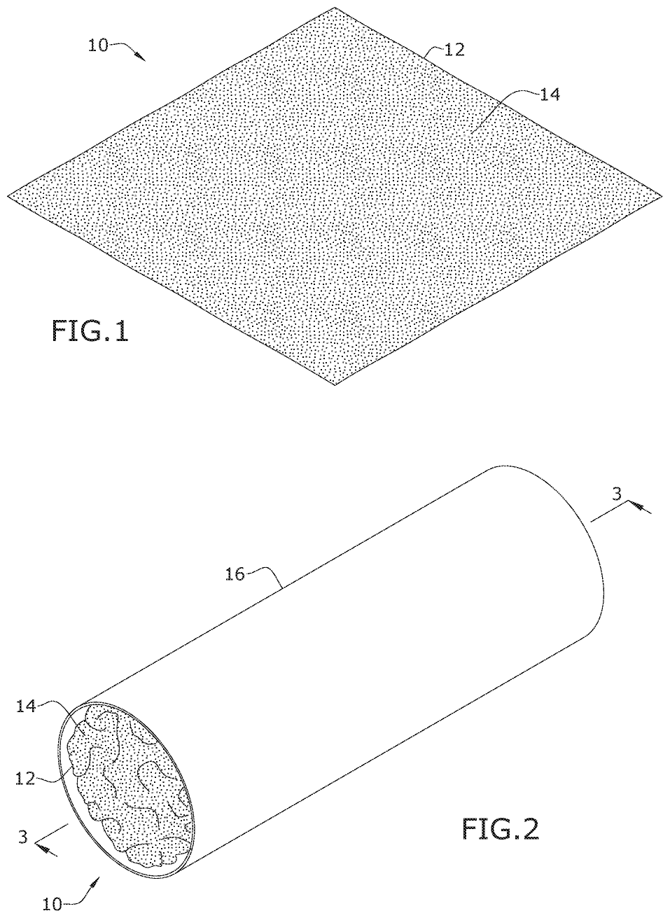

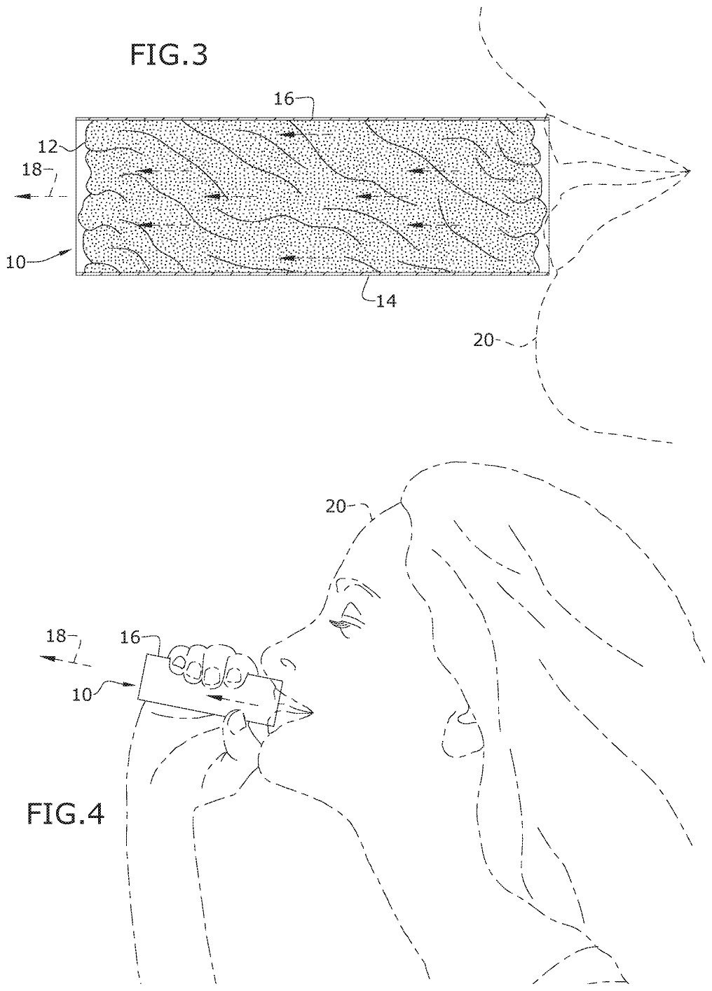

Referring to FIGS. 1 through 4, the present invention may include a mass of material 10. The mass of material 10 may be cheese cloth, hemp, bamboo material or the like. The material mass 10 may be in the form of a sheet 12, but is not necessarily limited to a sheet 12. Though in sheet form, the mass of material 10 may come in individual disposable scented sheets 12 approximately 8×8 inches (i.e. in a range between 4×4 and 12×12 inches), whereby the sheet 12 may be planar so that the thickness of the sheet 12 (or ply) is an eighth of an inch or less while the other two dimensions are approximately 8×8 inches. Or whatever dimensions lends a plurality of sheets to taking the form of a roll or in a stacked, interleaved form.

The mass of material 10 may be infused with essential oil 14. Essential oil 14 may be a concentrated hydrophobic liquid containing volatile chemical compounds from plants. Essential oils may include volatile oils, ethereal oils, aetherolea, or simply as the oil of the plant from which they were extracted, such as olive of clove. An essential oil is “essential” in the sense that it contains the “essence of” the plant's fragrance—the characteristic fragrance of the plant from which it is derived.

The present invention may embody a process whereby a user 20 puts an individual sheet 12 in a conduit 16, such as a roll or tube of their choice (in certain instances it could be a toilet paper roll) and blows smoke 18 through the conduit 16, as illustrated in FIG. 4, thereby removing or replacing the odor in the smoke with the odor of the essential oil 14. The user 20 can replace the sheet 12 of material mass 10 when the scent fades or as needed. The present invention may thus embody a disposable filtering agent sheet 12 infused with essential oils 14 to be used as a smoke 18 blow threw filter/air freshener for medical and recreational smokers that comes in a container (e.g., roll, box, or the like). The present invention may include sealed packaging along the container (not shown) to keep the essential oils 14 from evaporating. There can be a variety of scents infused to the sheets 12/material mass 10. Additionally, the present invention may be used as a deodorizer.

It should be understood, of course, that the foregoing relates to exemplary embodiments of the invention and that modifications may be made without departing from the spirit and scope of the invention as set forth in the following claims.

What is claimed is:

1. A method of rending an intrinsic smell of an exhale smoke odorless, comprising:
providing a material mass infused with essential oil;
packing said material mass in a conduit; and
exhaling through one end of the conduit.

2. The method of claim 1, wherein the material mass is cheese cloth, hemp, or bamboo.

3. The method of claim 2, wherein the material mass is dimensioned to be a sheet.

4. The method of claim 3, wherein the sheet is approximately eight by eight inches and has a thickness less than an eighth of an inch.

5. The method of claim 3, further comprising a plurality of sheets in a roll condition.

6. The method of claim 3, further comprising a plurality of sheets disposed in a stacked condition, wherein each two adjacent sheet are interleaved.

7. A refill element for a sploof for rendering an intrinsic smell of an exhale smoke odorless, comprising:
a material mass infused with essential oil.

8. The refill element of claim 7, wherein the material mass is cheese cloth, hemp, or bamboo.

9. The refill element of claim 8, wherein the material mass is dimensioned to be a sheet.

10. The refill element of claim 9, wherein the sheet is approximately eight by eight inches and has a thickness less than an eighth of an inch.

11. The refill element of claim 9, further comprising a plurality of sheets in a roll condition.

12. The refill element of claim 9, further comprising a plurality of sheets disposed in a stacked condition, wherein each two adjacent sheet are interleaved.
